(19) United States
(12) Patent Application Publication
(10) Pub. No.: US 2017/0289523 A1
(43) Pub. Date: Oct. 5, 2017

(54) DENTAL INTRAORAL SCANNER SYSTEM

(71) Applicants: VATECH Co., Ltd., Gyeonggi-do (KR); VATECH EWOO Holdings Co., Ltd., Gyeonggi-do (KR)

(72) Inventors: Jung LEE, Gyeonggi-do (KR); Jin Pyo CHUN, Gyeonggi-do (KR); Hyeong Jin JO, Gyeonggi-do (KR)

(73) Assignees: VATECH Co., Ltd., Gyeonggi-do (KR); VATECH EWOO Holdings Co., Ltd., Gyeonggi-do (KR)

(21) Appl. No.: 15/473,819

(22) Filed: Mar. 30, 2017

(30) Foreign Application Priority Data
Mar. 30, 2016 (KR) 10-2016-0038041

(51) Int. Cl. H04N 13/02; A61B 1/045; A61B 1/24; A61C 9/00; G06T 17/00; A61B 1/00

(52) U.S. Cl. CPC H04N 13/0207; H04N 13/0296; G06T 17/00; A61B 1/00006; A61B 1/00045; A61B 1/24; A61C 9/0053; A61B 1/00055; A61B 1/00039; A61B 1/045; H04N 2005/2255

(57) ABSTRACT

The present invention generally relates to a dental intraoral scanner system. In detail, the present invention includes: a scan unit sequentially imaging an intraoral structure in a scan mode; a control unit generating a three-dimensional modeling image for each scan mode by using the imaged intraoral structure; and a display unit displaying the three-dimensional modeling image, wherein the control unit switches the scan unit from a present scan mode to a following scan mode according to a user's command that is input through the scanning unit, or automatically switches from the present scan mode to the following scan mode when a three-dimensional modeling image of the present scan mode is completed.

### CROSS REFERENCE TO RELATED APPLICATION

The present application claims priority to Korean Patent Application No. 10-2016-0038041, filed Mar. 30, 2016, the entire contents of which is incorporated herein for all purposes by this reference.

### BACKGROUND ART

### Technical Field

The present invention generally relates to a dental intraoral scanner system. More particularly, the present invention relates to a dental intraoral scanner system capable of being switched to a scan mode for imaging an intraoral structure and of image processing by using an intraoral scanner.

### Description of the Related Art

Generally, in dental hospitals, structures of oral tissues such as the teeth of a patient are identified by performing impression-taking of teeth, and based on this, medical treatment is performed. However, the impression taking is problematic due to material consumption and cross infections. In addition, there is a possibility of breakage and inadequate preservation of the manufactured impression or gypsum model.

In addition, in order to identify structures of oral tissues, a conventional method that is widely used uses a two-dimensional projection image of an intraoral tissue structure obtained by inserting a sheet film or a digital sensor into the mouth and by irradiating the target with X-rays from the outside of the oral cavity. However, since the two-dimensional projection image shows a three-dimensional structure in a two-dimensional plane, errors such as distortion or unstitching may occur. In addition, since radiation is applied to the patient, it may cause medical problems and financially burden the patient. Furthermore, use of the above method is complicated.

Recently, in order to obtain three-dimensional data of structures of oral tissues, a dental intraoral scanning system that implements a three-dimensional modeling image of a structure of an oral tissue by using measuring light such as a laser is widely used. A general dental intraoral scanning system includes: an intraoral scanner held by a user to image a structure of an oral tissue; and a PC connected to the intraoral scanner in a wired manner to generate a three-dimensional modeling image as a result of imaging.

In one embodiment, processes of obtaining a three-dimensional modeling image of a target tooth by using a dental intraoral scanning system for the manufacture of prostheses, etc. is briefly described below.

First, in a first scan mode, a prosthetic treatment target tooth that is at least a part of one of an upper jaw or a lower jaw, and left and right peripheral teeth of the target tooth are imaged. Accordingly, a three-dimensional modeling image of the first scan mode is obtained. Next, in a second scan mode, an occlusal tooth that occludes with the target tooth and is at least a part of the remaining one of the upper jaw or the lower jaw, and left and right peripheral teeth of the occlusal tooth are imaged, and three-dimensional data thereof is obtained. Accordingly, a three-dimensional modeling image of the second scan mode is obtained. Then, in a third scan mode, the target tooth being at least a part of the upper and lower occlusal state and the left and right peripheral teeth of the target tooth, and the occluded tooth and the left and right peripheral teeth of the occluded tooth are imaged, and three-dimensional data thereof is obtained. Accordingly, a three-dimensional modeling image of the third scan mode is obtained.

Then, the three-dimensional modeling images of the first and second scan modes are aligned based on the three-dimensional modeling image of the third scan mode, and the entire three-dimensional modeling image of the first and second scan modes that are aligned with each other becomes a final three-dimensional modeling image.

However, in the conventional dental intraoral scanning system, the user has to directly handle the intraoral scanner and the PC alternately, for example, the user has to directly manipulate the PC to switch the intraoral scanner to the first, second, and third scan modes. In addition, the user has to directly manipulate the PC to check the three-dimensional modeling images of the first, second, and third scan modes. In addition, the user has to directly manipulate the PC to re-image when the three-dimensional modeling images of the first, second, and third scan modes are abnormal.

Therefore, the overall operation time increases and there is also a problem that secondary infection may occur due to cross-contact between the intraoral scanner and a device such as PC, since the user manipulates the PC with his or her hand and operates the intraoral scanner again with that hand.

The foregoing is intended merely to aid in the understanding of the background of the present invention, and is not intended to mean that the present invention falls within the purview of the related art that is already known to those skilled in the art.

### Documents of Related Art

(Patent Document 1) Korean Patent No. 10-1176770;

(Patent Document 2) Japanese Patent Application

### SUMMARY OF INVENTION

Accordingly, the present invention has been made keeping in mind the above problems occurring in the related art, and the present invention is intended to propose an intraoral scanner system in which scan time is reduced by correcting in real time errors that are generated while imaging, and mode switching and image processing are made available from the. Thereby, overall operations may be made easy and convenient, and at the same time, secondary infections due to a cross contact between the intraoral scanner and a device such as PC may be prevented.

In order to achieve the above object, according to one aspect of the present invention, there is provided an intraoral scanner system including: a scan unit sequentially imaging an intraoral structure in a scan mode; a control unit generating a three-dimensional modeling image for each scan mode by using the imaged intraoral structure; and a display unit displaying the three-dimensional modeling image, wherein the control unit switches the scan unit from a present scan mode to a following scan mode according to a user's command that is input through the scanning unit, or automatically switches from the present scan mode to the following scan mode when a three-dimensional modeling image of the present scan mode is completed.

Herein, the scan mode may include: a first scan mode that images at least a part of one of an upper jaw or a lower jaw; a second scan mode that images at least a part of the remaining one of the upper jaw or the lower jaw; and a third scan mode that images at least a part of an occlusion state of the upper jaw and the lower jaw.

In addition, the apparatus may further include: an error detection unit detecting a scan error of the three-dimensional modeling image for each scan mode and displaying the detected scan error on the display unit, and wherein the control unit may switch of the scan mode of the scan unit when the scan error of the three-dimensional modeling image is not detected.

In addition, the apparatus may further include: a button provided in one side of the scan unit and through which the user's command is input.

In addition, the apparatus may further include: a scan range setting unit setting a scan range for each scan mode, and wherein the control unit may switch the scan mode when imaging for the scan range of each scan mode is completed.

In addition, when the three-dimensional modeling image for each scan mode is completed, the control unit may switch off the scan unit to a remote mode.

In addition, the scan unit may include at least one motion detection sensor, and the control unit may change at least one of a position or a direction of the three-dimensional modeling image for each scan mode that is displayed on the display unit according to a detection result of the motion detection sensor while the scan unit operating in the remote mode.

An intraoral scanner system according to the present invention may reduce imaging time by correcting in real time errors that are generated while imaging. Since the intraoral scanner system allows mode switching and image processing of a three-dimensional modeling image by using the intraoral scanner, the overall operations may be made simple and convenient. In addition, since there is no cross contact between the intraoral scanner and a device such as a PC, it has an effect of preventing secondary infection.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a view showing an intraoral scanner system according to an embodiment of the present invention;

FIGS. 2A and 2B are views showing a scan unit according to the embodiment of the present invention;

FIGS. 3A and 3B are views showing a display unit according the embodiment of the present invention;

FIG. 4 is a view showing a remote mode of the intraoral scanner system according the embodiment of the present invention;

FIGS. 5A and 5B are view showing a scan range setting unit according the embodiment of the present invention;

FIG. 6 is a view showing an operating method of the intraoral scanner system according to the embodiment of the present invention; and

FIG. 7 is a view showing a scan unit according to the embodiment of the present invention.

### DESCRIPTION OF EMBODIMENTS

Embodiments of the present disclosure will now be described in detail with reference to the accompanying drawings. The disclosure may, however, be exemplified in many different forms and should not be construed as being limited to the specific embodiments set forth herein. In the drawings, the shapes and sizes of elements may be exaggerated for the sake of clearer description. Reference now should be made to the drawings, in which the same reference numerals are used throughout the different drawings to designate the same or similar components.

Through the whole document, the term “connected to” or “coupled to” that is used to designate a connection or coupling of one element to another element includes both a case that an element is “directly connected or coupled to” another element and a case that an element is “electronically connected or coupled to” another element via still another element. Also, when a part may “include or comprise” a certain constituent element, unless specified otherwise, it may not be construed to exclude another constituent element but may be construed to further include other constituent elements.

In addition, terms “first”, “second”, etc. are used to distinguish an element from other elements, and the scope of rights will not be limited by the foregoing terms. For example, a first element may be named as a second element, and similarly, the second element may be named as the first element.

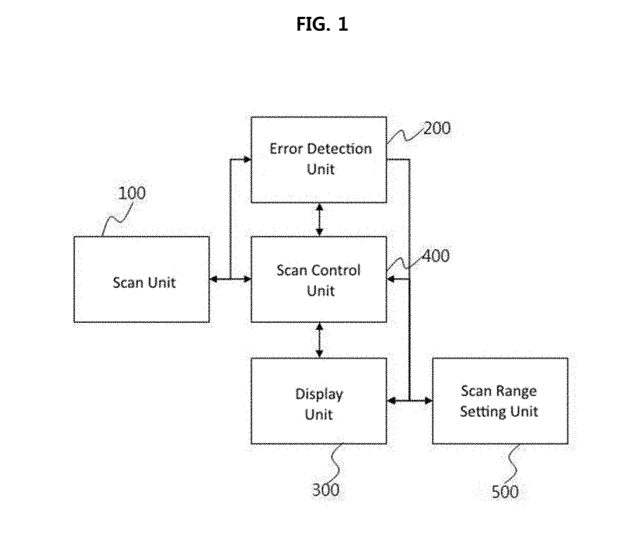

FIG. 1 is a view showing an intraoral scanner system according to an embodiment of the present invention, the intraoral scanner system includes: a scan unit 100; an error detection unit 200; a display unit 300; a control unit 400; and a scan range setting unit 500.

The scan unit 100 is a part that a user directly holds to image an intraoral structure to obtain a three-dimensional modeling image. The scan unit 100 obtains image data in a frame unit from the intraoral structure and transmits the obtained image data to the control unit 400

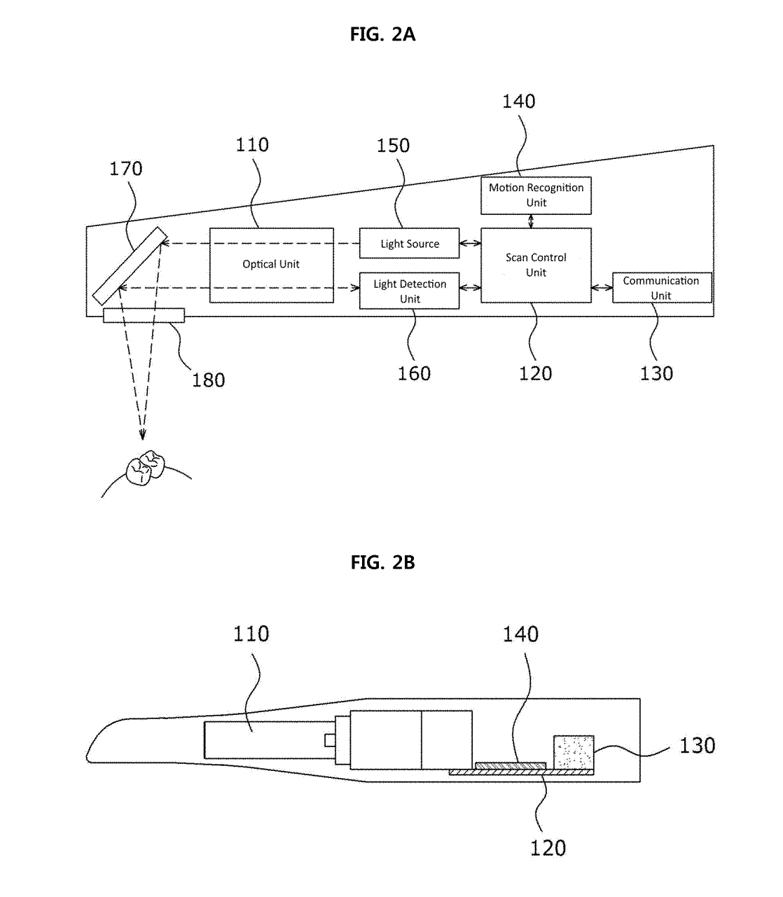

FIG. 2A is a block diagram showing an embodiment of the scan unit 100, and FIG. 2B is a view schematically showing an example of the scan unit 100. The scan unit 100 includes an optical unit 110, a scan control unit 120, a communication unit 130, a motion recognition unit 140, a light source 150, and a light detection unit 160. FIG. 2B is a view showing an arrangement structure of the motion recognition unit 140, and other components are omitted.

The optical unit 110 may include at least one lens, and at least one of the lenses may be a water lens (not shown). The optical unit 110 optically adjusts output light irradiated from the light source 150 controlled by the scan control unit 120 so that the output light focuses on a reflection plate 170, and optically adjusts reflected light that is reflected from the intraoral structure so that the reflected light focuses on the light detection unit 160. The output light focused on the reflection plate 170 is irradiated to the intraoral structure by passing a transmission window 180, and the reflected light that is reflected from the intraoral structure is focused on the optical unit 110 by passing the transmission window 180 and the reflection plate 170.

The light source 150 may be a projector, a laser diode, a light emitting diode (LED), etc. that irradiates output light, and the output light may include a predetermined pattern.

The light detection unit 160 may be an optical sensor that generates image signals by detecting reflected light. The optical sensor may be a photo-detector such as complementary metal-oxide semiconductor (CMOS), charged coupled device (CCD), position sensitive device (PDS), etc., but it is not limited thereto.

The scan control unit 120 irradiates output light by controlling the light source 150, outputs a trigger signal to the light detection unit 160 to grab an image signal of the light detection unit 160 so that the scan control unit 120 receives grabbed image data. In addition, the scan control unit 120 generates captured image data for at least a part of a two-dimensional image among the image data, while generating modeling image data for a three-dimensional modeling image by properly sampling the image data. In addition, the scan control unit 120 transmits the captured image data and the modeling image data to the control unit 400 by using the communication unit 130. Herein, the captured image data and the modeling image data may be generated by the control unit 400. For reference, the scan control unit 120 may be connected to the light detection unit 160 by using an additional camera trigger cable to output the trigger signal to the light detection unit 160

The captured image data transmitted through the communication unit 130 is input to the control unit 400 and processed in a two-dimensional image that shows an imaging process. The modeling image data is processed in a three-dimensional modeling image that includes depth data of the intraoral structure, in other words, three-dimensional data. Herein, the communication unit 130 may also serve a power supply function, and bi-directionally transmit various control signals between the scan unit 100 and the control unit 400. As the communication unit 130, a general wireless or wired communication module that provides a function of bi-directionally transmitting image data, control signals, and power between the unit 400 and the scan unit 100 may be widely used. For example, when the control unit 400 is provided in a

PC that includes Window 7 as an operating system, a universal serial bus (USB) 2.0 may be used for the communication unit 130.

The motion recognition unit 140 is a sensor capable of detecting a motion of the scan unit 100, and includes at least one of a gyro sensor and an acceleration sensor. The motion recognition unit 140 generates a motion recognition signal according to the motion of the scan unit 100 and transmits the generated motion recognition signal to the control unit 400 through the communication unit 130. Herein, the motion recognition signal is input to the control unit 400 as a user's command used in a remote mode that will be described later.

Meanwhile, as scan modes that image a structure of an intraoral tissue, the scan unit 100 performs a first scan mode that images at least a part of one of an upper jaw or a lower jaw, a second scan mode that images at least a part of the remaining one of the upper jaw or the lower jaw, and a third scan mode that images at least a part of an occlusion state of the upper jaw or the lower jaw. Therefore, the control unit 400 generates a three-dimensional modeling image for each scan mode.

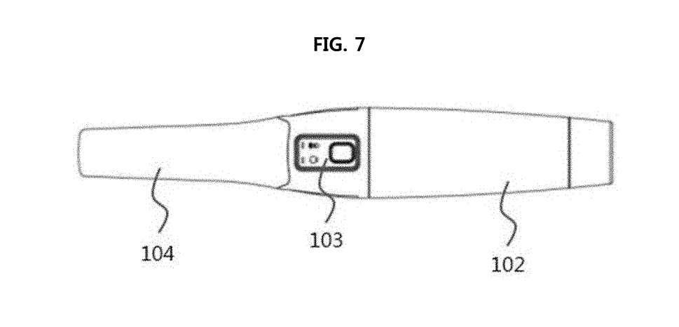

The scan unit 100 may include an additional button (refer to 103 of FIG. 7) for switching of the plurality of the scan modes. The control unit 400 may switch the scan mode of the scan unit 100 according to a user's command that is input through the button. In other words, when the user presses the button after imaging using the first scan mode is completed, the unit 400 switches the scan unit 100 from the first scan mode to the second scan mode. In addition, when the user presses the button after imaging using the second scan mode is completed, the unit 400 switches the scan unit 100 from the second scan mode to the third scan mode. In addition, when the user presses the button after imaging using the third scan mode is completed, the control unit 400 finishes imaging using the third scan mode. As a result, the intraoral scanner system according to the embodiment of the present invention may be operated in the first, second, and third scan modes by manipulating the scan unit 100 and without the user manipulating external devices such as a PC, etc.

Alternatively, the plurality of scan modes of the scan unit 100 may be automatically switched. Herein, the control unit 400 determines whether or not the scan mode is switched off. For example, when a generation of a three-dimensional modeling image of the first scan mode is completed, the control unit 400 may inform the user that the first scan mode is completed through the display unit 300 that will be described later, and switch the scan unit 100 from the first scan mode to the second scan mode. The control unit 400 switches the scan unit 100 from the second scan mode to the third scan mode by using the same methods.

In addition, the scan unit 100 may operate in a remote mode that transmits a user's command used for image processing of various images displayed on the display unit 300 according to a motion of the scan unit 100. As a result, the intraoral scanner system according to the embodiment of the present invention may enable image processing by use the scan unit 100 without a user's manipulation of external devices such as PC, etc. In other words, in the remote mode, the scan unit 100 becomes an object for inputting a user's command, and a motion of the scan unit 100 detected by the motion recognition unit 140 is transmitted to the control unit 400 and used as the user's command.

Herein, the scan unit 100 may include an additional button for switching from the scan mode to the remote mode.

FIG. 7 is a view showing the scan unit 100 according to the embodiment of the present invention. The scan unit 100 includes a body 102 that the user holds by hand, and a probe 104 extending from a distal end of the body 102 and inserted into the mouth of an examinee. The scan unit 100 may further include an additional button 103 provided on the distal end of the body where a user places his/her thumb in body 102 is held by hand. Therefore, the user may select the scan mode or the remote mode by pressing the button 103.

Alternatively, the scan mode and the remote mode may be automatically switched by the control unit 400. For example, the control unit 400 may calculate a distance between the intraoral structure and the scan unit 100 by using image data obtained by the scan unit 100, when the calculated distance is equal to or less than a predetermined distance, for example, equal to or less than 7 mm, the control unit 400 may switch the scan unit 100 to the scan mode. Alternatively, when the calculated distance exceeds 7 mm, the control unit 400 may switch the scan unit 100 to the remote mode. Therefore, the user may select the remote mode by pulling the scan unit 100 out of the patient's mouth or away from the intraoral structure, and select the scan mode by performing the opposite operations.

Referring again to FIG. 1, the error detection unit 200 detects a scan error in real time while the control unit 400 generates the three-dimensional modeling image, and generates and displays scan error data on the display unit 300 when the scan error is detected.

Herein, the scan error may include non-scan, scan distortion, and duplicated scan. The error detection unit 200 may detect the scan error in real time by comparing a predetermined base three-dimensional modeling image with the three-dimensional modeling image generated by the control unit 400 in real time. For example, the error detection unit 200 may generate differential data between the base three-dimensional modeling image and the three-dimensional modeling image generated by the control unit 400, and determine that the scan error has occurred when a value of the differential data exceeds a predetermined stored tolerance value. When the scan error is detected as described above, the scan error is displayed on the display unit 300 in real-time and informed to the user.

The display unit 300 displays the two-dimensional image and the three-dimensional modeling image generated by the control unit 400, and displays the scan error data on the three-dimensional modeling image received from the error detection unit 200.

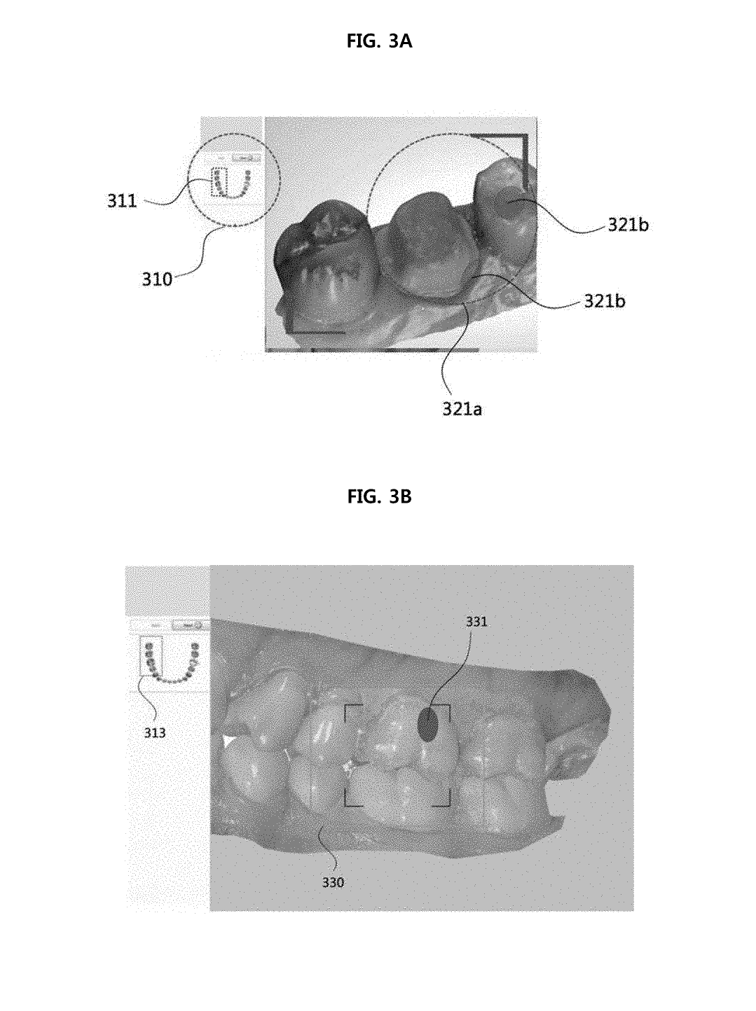

FIGS. 3A and 3B are views showing the display unit 300 according the embodiment of the present invention. In the first, second, and third scan modes, the display unit 300 displays a three-dimensional modeling image for each scan mode. In addition, the display unit 300 may display the two-dimensional image through an additional window, and display a part 311 in which imaging is processed as a two-dimensional arch model 310.

Herein, the error detection unit 200 may widely display a part in which the scan error has occurred in a shape of a FIG. 312a in the three-dimensional modeling image for each scan mode, or may display the part in which the scan error has occurred in a specific color 321b in the three-dimensional modeling image. Therefore, the user may check the scan error parts 321a and 321b in real-time through the display unit 300, and perform re-scanning a part where the scan error has occurred for each scan mode to solve the error.

In addition, the control unit 400 may re-image a corresponding part when the scan unit 100 moves to a position associated with the scan error part among the intraoral structure, and correct the scan error part by using image data obtained through re-imaging. Meanwhile, the first, second, and third scan modes may be switched by using the button of the scan unit 100, or may be automatically switched by the control unit 400. The control unit 400 may switch the scan unit 100 to the second scan mode by a button input of the scan unit 100 or may automatically switch the scan unit 100 to the second scan mode when the three-dimensional modeling image of the first scan mode has no scan error, or a scan error thereof is resolved. Then, the control unit 400 may switch the scan unit 100 to the third scan mode by a button input of the scan unit 100 or may automatically switch the scan unit 100 to the third scan mode when the three-dimensional modeling image of the second scan mode has no scan error, or a scan error thereof is solved. Then, the control unit 400 may terminate the scan mode and switch the scan unit 100 to the remote mode by a button input of the scan unit 100 or may automatically terminate the scan mode and switch the scan unit 100 to the remote mode when the three-dimensional modeling image of the third scan mode has no scan error, or a scan error thereof is solved.

In addition, in the remote mode, the display unit 300 displays a predetermined interface tool for image processing the three-dimensional modeling images of the first, second, and third scan modes, and aligns the three-dimensional modeling images of the first and second scan modes based on the three-dimensional modeling image of the third scan mode according to a user's manipulation.

In the remote mode, the scan unit 100 becomes an object of a user's manipulation for image processing of the three-dimensional modeling image, the motion recognition unit 140 recognizes a motion of the scan unit 100 and transmits a user's command associated therewith to the control unit 400, and the control unit 400 completes a final three-dimensional modeling image by performing image processing such as moving, rotating, etc. the three-dimensional modeling images of the first, second and third scan modes depending on the user's command according to the motion of the scan unit 100.

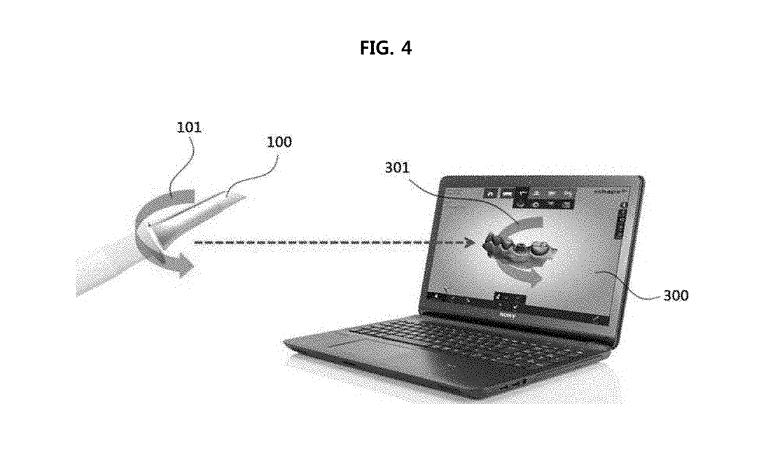

FIG. 4 is a view showing the remote mode according the embodiment of the present invention. When the scan unit 100 rotates in a predetermined direction 101, the three-dimensional modeling image displayed on the display unit 300 also rotates in an associated direction 301. In addition, according to a motion recognition signal of the motion recognition unit 140, the control unit 400 may properly perform image processing of the three-dimensional modeling image according to the motion of scanning unit 100 such as zooming in the three-dimensional modeling image when the control unit 400 determines that the scan unit 100 approaches the display unit 300, zooming out the three-dimensional modeling image when the control unit 400 determines that the scan unit 100 is far away from the display unit 300, and moving the three-dimensional modeling image to an associated position when the control unit 400 determines that the scan unit 100 has moved, etc.

Referring again to FIG. 1, the user may designate a starting point and an end point of imaging including a position of a target teeth by using the scan range setting unit 500 so that the control unit 400 may recognize a scan range for each scan mode. For this, the scan range setting unit 500 may display a predetermined interface screen on the display unit 300, and the user may designate the scan range through the predetermined interface screen. For example, the scan range may be designated in a tooth unit, and the scan range designated by the user is transmitted to the control unit 400.

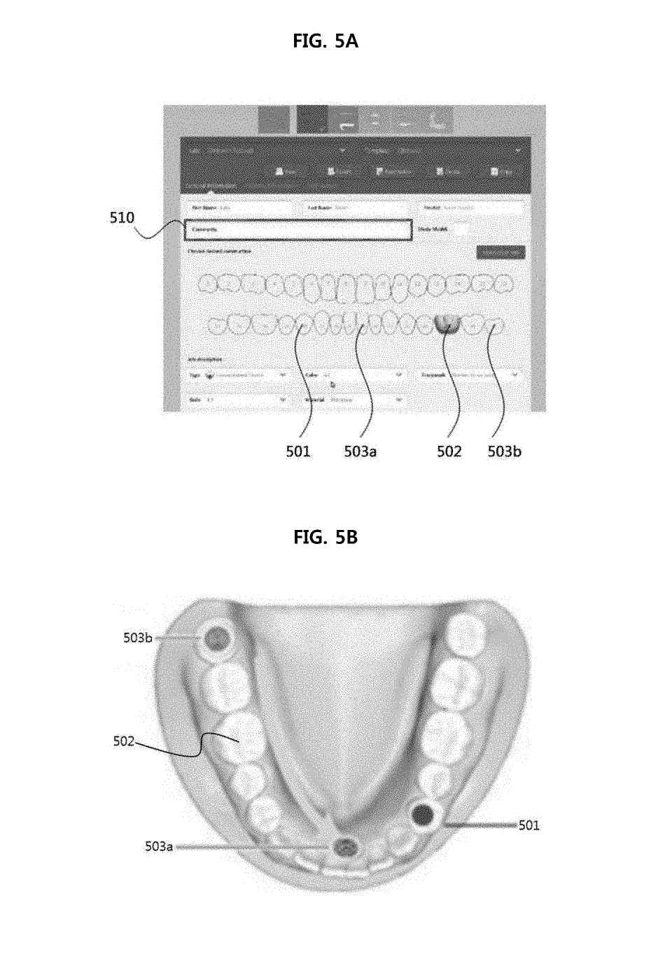

FIGS. 5A and 5B are views respectively showing the interface screen of the scan range setting unit 500 according to the embodiment of the present invention.

As shown in FIG. 5A, the scan range setting unit 500 may display upper teeth and lower teeth in sequence by using the display unit 300, and the user designates a scan range, in other words, a starting point and an end point of imaging, by selecting a target tooth and right and left peripheral teeth thereof in the teeth of the upper jaw or the lower jaw. For reference, designating of the scan range through the scan range setting unit 500 is normally performed by using an additional input device before selecting the scan mode, but if necessary, it may be understood without any further explanation that designating of the scan range may be performed by using the scan unit 100.

Herein, as shown in FIG. 5A, the user may set a scan end tooth 501, a scan target tooth 502, first and second scan guide teeth 503a and 503b in the teeth of the upper jaw or the lower jaw that are displayed on display unit 300. In addition, in the scan mode, the control unit 400 may generate a three-dimensional modeling image by imaging of the scan unit 100, complete a three-dimensional modeling image of a corresponding scan mode by using a three-dimensional modeling image between the scan guide teeth 503a and 503b including the scan target tooth 502 when image data of the scan end tooth 501 is detected, and switch the scan unit 100 to a next scan mode or terminate the scan mode of the scan unit 100 and switch the scan unit 100 to the remote mode assuming that there is no scan error part.

In addition, the scan range setting unit 500 may provide a window within the interface screen or separately therefrom, and the user may select or designate the scan end tooth 501, the scan target tooth 502, and the first and second guide teeth 503a and 503b by inputting numbers of the teeth in the window. For reference, FIG. 5B shows only the lower jaw for convenience, but it is also possible to designate the scan range for the upper jaw and for the occlusion in the same manner. Alternatively, the user may designate only a specific target tooth in the teeth of the upper jaw or the lower jaw. Herein, the scan range setting unit 500 may set the scan range by designating a predetermined number of left and right peripheral teeth of the target tooth and an occlusal tooth.

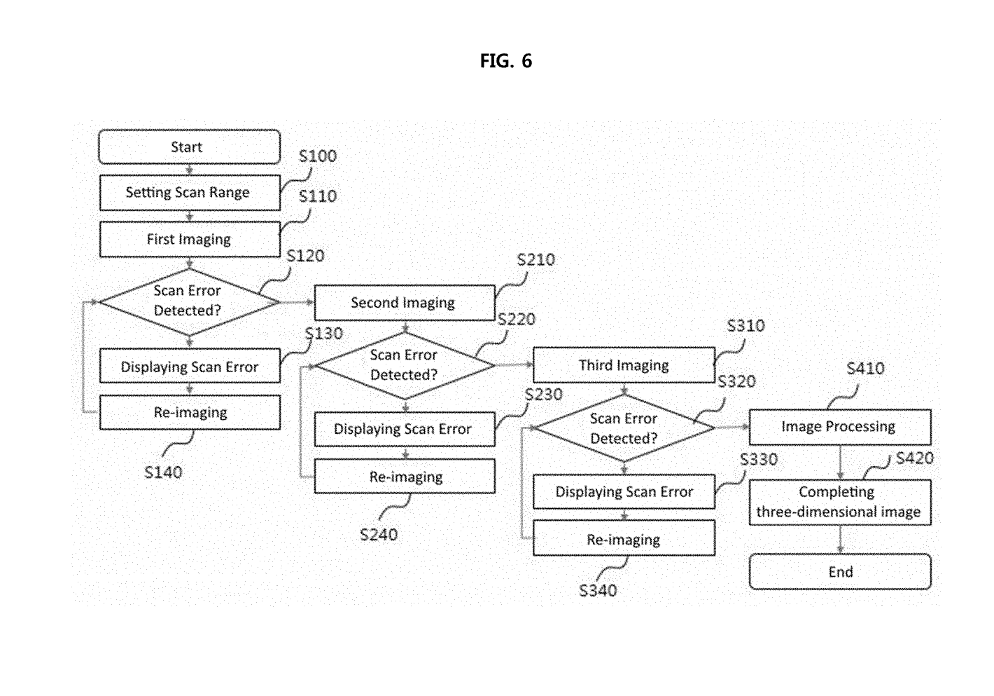

FIG. 6 is a view showing an operating method of the intraoral scanner system according to the embodiment of the present invention and the method will be described with reference to FIGS. 1 to 5.

First, in step S100, a user sets a scan range through the interface screen of the scan range setting unit 500 displayed on the display unit 300, and the scan range setting unit 500 transmits the scan range set by the user to the control unit 400

Then, in steps S110, S120, and S130, the user images an intraoral structure of a patient by using the scan unit 100. The control unit 400 sequentially generates a three-dimensional modeling image for each scan mode, and displays the generated three-dimensional modeling images on the display unit 300.

In detail, in step S110, the user images either the upper jaw or the lower jaw, for example, a part of the lower jaw, by using the scan unit 100 in a first scan mode.

Accordingly, in steps S120, S130, and S140, the control unit 400 generates a three-dimensional modeling image of the part of the lower jaw and displays the generated image on the display unit 300, the error detection unit 200 displays a scan error when the scan error is detected while the control unit 400 generates the three-dimensional modeling image of the first scan mode, the user re-images a scan error part to resolve the scan error in real-time.

When an error-free three-dimensional modeling image of the scan range of the lower jaw is obtained by the above steps, the scan mode is switched to the second scan mode. Switching from the first scan mode to the second scan mode may be performed by a button input of the scan unit 100 or may be automatically performed by the control unit 400.

Next, in step S210, the user images the remaining one of the upper jaw or the lower jaw, for example, a part of the upper jaw, by using the scan unit 100 in the second scan mode.

Accordingly, in steps S220, S230, and S240, the control unit 400 generates a three-dimensional modeling image of the part of the upper jaw and displays the generated image on the display unit 300, the error detection unit 200 displays a scan error when the scan error is detected while the control unit 400 generates the three-dimensional modeling image of the second scan mode, the user re-images a scan error part to resolve the scan error in real-time.

When a three-dimensional modeling image of second imaging is obtained by the above steps, the scan mode is switched to the third scan mode. Switching from the second scan mode to the third scan mode may be performed by a button input of the scan unit 100 or may be automatically performed by the control unit 400. In step S310, the user images a part of an occlusion state of teeth by using scan unit 100 in the third scan mode and by using the same methods.

Accordingly, in steps S320, S330, and S340, the control unit 400 generates a three-dimensional modeling image of the part of the occlusion state and displays the generated image of the third scan mode on the display unit 300, the error detection unit 200 displays a scan error when the scan error is detected while the control unit 400 generates the three-dimensional modeling image of the third scan mode, the user re-images a scan error part to resolve the scan error in real-time.

When the first, second, and third scan modes are completed by the above steps, the scan mode is switched to a remote mode.

In steps S410 and S420, in the remote mode, the display unit 300 displays a predetermined interface tool and the three-dimensional modeling images of the first, second, and third scan modes, and the user aligns the three-dimensional modeling images of the first and second scan modes based on the three-dimensional modeling image of the third scan mode by using scan unit 100. Herein, the user operates the scan unit 100 according to a predefined rule, the motion recognition unit 140 recognizes a motion of the scan unit 100 and transmits a user's command associated therewith to the control unit 400, and the control unit 400 completes a final three-dimensional modeling image by performing image processing such as moving, rotating, etc. the three-dimensional modeling images of the first, second, and third scan modes depending on the user's command associated with the motion of the scan unit 100.

Herein, the scan unit 100 may include an additional light emitting diode (LED) (not shown), the LED may emit a current state, for example, white color light when the scan unit 110 operates in the scan mode, or, for example, green color light when the scan unit 110 operates in the remote mode.

Meanwhile, the error detection unit 200, the control unit 400, the display unit 300, the scan range setting unit 500 may be composed of a single computer or a plurality of computers, and although not shown, may include an input unit, a storage unit, etc.

The input unit may be configured with a hardware and software module to receive a user's command apart from the scan unit 100. The input unit may be used for inputting various required commands to the control unit 400, for inputting various images to the storage unit, for indicating a part or the entire part of images displayed on the display unit 300 to perform various image processing. In one embodiment, the input unit may include a keyboard, a touchpad, a keypad, a mouse, etc., but it is not limited thereto. For example, the input unit may include a graphic user interface (GUI) controlled by using the above input devices.

The display unit 300 may be used for displaying images generated according to various embodiments of the present invention, and include various displays such as LCD display, LED display, AMOLED display, CRT display, etc.

The storage unit may be used for storing intermediate result image data obtained by performing image processing according to various embodiments of the present invention such as three-dimensional modeling images of the first, second, and third scan modes, a final three-dimensional modeling image, etc., for storing result image data obtained by performing image processing according to various embodiments of the present invention, and for storing parameter values that are necessary for performing image processing according to various embodiments of the present invention. The storage unit may further include a software/firmware necessary for implementing the control unit 400, etc. The storage unit may be implemented as a storage medium of any one a memory of flash memory type, hard disk type, a multi media card (MMC) type, a card type (for example, secure digital (SD), eXtream Digital (XD), etc.), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk, etc., but it is not limited thereto. It will be appreciated by those skilled in the art that implementations of the storage unit are not limited thereto.

The error detection unit 200, the control unit 400, scan range setting unit 500, as a functional classification and in terms of hardware, may be implemented by suing at least one of application specific integrated circuits (ASICs), digital signal processors (DSPs), digital signal processing devices (DSPDs), programmable logic devices (PLDs), field-programmable gate arrays (FPGAs), processors, controllers, micro-controllers, and microprocessors. In addition, they may be implemented as firmware/software modules executable on the above hardware platform. Herein, the firmware/software modules may be implemented by one or more software applications written in a suitable program language.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. An intraoral scanner system comprising:
a scan unit sequentially imaging an intraoral structure in a scan mode;
a control unit generating a three-dimensional modeling image for each scan mode by using the imaged intraoral structure; and
a display unit displaying the three-dimensional modeling image,
wherein the control unit switches the scan unit from a present scan mode to a following scan mode according to a user's command that is input through the scanning unit, or automatically switches from the present scan mode to the following scan mode when a three-dimensional modeling image of the present scan mode is completed.

2. The apparatus of claim 1, wherein the scan mode includes:
a first scan mode that images at least a part of one of an upper jaw or a lower jaw;
a second scan mode that images at least a part of the remaining one of the upper jaw or the lower jaw; and
a third scan mode that images at least a part of an occlusion state of the upper jaw and the lower jaw.

3. The apparatus of claim 1, further comprising:
an error detection unit detecting a scan error of the three-dimensional modeling image for each scan mode and displaying the detected scan error on the display unit, and
wherein the control unit switches of the scan mode of the scan unit when the scan error of the three-dimensional modeling image is not detected.

4. The apparatus of claims 1, further comprising:
a button provided in one side of the scan unit and through which the user's command is input.

5. The apparatus of claims 1, further comprising:
a scan range setting unit setting a scan range for each scan mode, and
wherein the control unit switches the scan mode when imaging for the scan range of each scan mode is completed.

6. The apparatus of claim 5, wherein when the three-dimensional modeling image for each scan mode is completed, the control unit switches off the scan unit to a remote mode.

7. The apparatus of claim 6, wherein the scan unit includes at least one motion detection sensor, and the control unit changes at least one of a position or a direction of the three-dimensional modeling image for each scan mode that is displayed on the display unit according to a detection result of the motion detection sensor while the scan unit operating in the remote mode.
